(19) United States
(12) Patent Application Publication
(10) Pub. No.: US 2023/0033099 A1
(43) Pub. Date: Feb. 2, 2023

(54) Dendritic Cells Pulsed With Tumor Membrane Vesicles And Uses In Treating Cancer

(71) Applicant: Emory University, Atlanta, GA (US)

(72) Inventors: Periasamy Selvaraj, Lilburn, GA (US); Ramireddy Bommireddy, Johns Creek, GA (US); Luis Enrique Munoz, Decatur, GA (US); Christopher D. Pack, Atlanta, GA (US); Jakkidi Chandra Shaker Reddy, Roswell, GA (US); Sampath Ramachandiran, Duluth, GA (US)

(21) Appl. No.: 17/876,413

(22) Filed: Jul. 28, 2022

Related U.S. Application Data
(60) Provisional application No. 63/226,401, filed on Jul. 28, 2021.

(51) Int. Cl. A61K 39/00; A61P 35/00

(52) U.S. Cl. CPC A61K 39/001106; A61P 35/00; A61K 2039/5158; A61K 2039/5154; A61K 2039/812

(57) ABSTRACT

This disclosure relates to methods of treating cancer using dendritic cells pulsed with tumor membrane vesicles as disclosed herein. In certain embodiments, the tumor membrane vesicles contain fusion proteins with a cytokine and a glycosyl phosphatidylinositol domain. In certain embodiments, the cytokine is granulocyte-macrophage colony-stimulating factor (GM-CSF). In certain embodiments, tumor membrane vesicles contain fusion proteins with IL-12 and a glycosyl phosphatidylinositol domain.

### CROSS-REFERENCE TO RELATED APPLICATIONS

This application claims the benefit of U.S. Provisional Application No. 63/226,401 filed Jul. 28, 2021. The entirety of this application is hereby incorporated by reference for all purposes.

### STATEMENT REGARDING FEDERALLY SPONSORED RESEARCH OR DEVELOPMENT

This invention was made with government support under CA202763 awarded by the National Institutes of Health. The government has certain rights in the invention.

### BACKGROUND

Methods for treating cancer are not universally effective, and it is common for patients to relapse after therapeutic treatments. Dendritic cells (DCs) are antigen presenting immune cells that facilitate T cell responses to clear cancerous cells. Sipuleucel-T is an FDA approved DC-based immunotherapy for prostate cancer which utilizes a fusion protein to stimulate DCs ex vivo with GM-CSF and simultaneously deliver the antigen PAP. This approach is restricted by the breadth of immunity elicited to a single antigen, and to cancers that have a defined tumor associated antigen. Thus, there is a need to identify improved therapeutic strategies.

One particular type of breast cancer, triple negative breast cancer (TNBC), afflicts thousands of women each year in the US, typically at a younger age than other breast cancers. Even with conventional radiation and chemotherapy regimens, patients have poor prognosis, experiencing early, frequent relapses in comparison to other breast cancers. A high level of intratumoral as well as patient-to-patient heterogeneity is observed among triple negative patients, making it even more difficult to treat. See Gerlinger et. al., The New England Journal of Medicine, 366:883-92 (2012). Therapies effective for other cancers, even other breast cancers, frequently prove ineffective at treating TNBC. TNBC is a clear area of significant unmet medical need, and new therapies that address patient-to-patient variation in tumor targets are needed.

Patel et al. report plasma membrane vesicles decorated with glycolipid-anchored antigens and adjuvants via protein transfer as an antigen delivery platform for inhibition of tumor growth. Biomaterials, 2016, 74, 231-44.

Mittal et al. report an emerging role of extracellular vesicles in immune regulation and cancer progression. Cancers, 2020, 12, 3563.

Crescitelli et al. report the isolation and characterization of extracellular vesicle subpopulations from tissues. Nat Protoc, 2021, 16(3):1548-1580.

Munoz et al. report dendritic cells pulsed with cytokine-adjuvanted tumor membrane vesicles inhibit tumor growth in HER2-positive and triple negative breast cancer models. Int J Mol Sci, 2021, 22, 8377. See also US 2015/0071987 and US 2018/0128833.

References cited herein are not an admission of prior art.

### SUMMARY

This disclosure relates to methods of treating cancer using dendritic cells pulsed with tumor membrane vesicles as disclosed herein. In certain embodiments, the tumor membrane vesicles contain fusion proteins with a cytokine and a glycosyl phosphatidylinositol domain. In certain embodiments, the cytokine is granulocyte-macrophage colony-stimulating factor (GM-CSF). In certain embodiments, tumor membrane vesicles contain fusion proteins with IL-12 and a glycosyl phosphatidylinositol domain.

In certain embodiments, this disclosure relates to methods of treating cancer comprising administering an effective amount of dendritic cells pulsed with tumor membrane vesicles as disclosed herein. In certain embodiments, tumor membrane vesicles contain fusion proteins with a cytokine surface protein linked to glycosyl phosphatidylinositol domain; thus, resulting in dendritic cells that contain the cytokine surface protein(s). In certain embodiments, the cytokine is granulocyte-macrophage colony-stimulating factor (GM-CSF).

In certain embodiments, this disclosure relates to dendritic cells pulsed in vitro with tumor membrane vesicles comprising: a first fusion protein on the surface of the tumor membrane vesicles having a granulocyte-macrophage colony-stimulating factor (GM-C SF) and a glycosyl phosphatidylinositol domain and a second fusion protein on the surface of the tumor membrane vesicles having interleukin 12 and a glycosyl phosphatidylinositol domain. In certain embodiments, the dendritic cells further comprise a tumor cell antigen on the surface derived from the tumor membrane vesicles.

In certain embodiments, the tumor membrane vesicles (TMV) are obtained from a tumor tissue or cultured tumor cells. In certain embodiments, the tumor membrane vesicles (TMV) contain a third fusion protein comprising a cancer antigen and a glycosyl phosphatidylinositol domain.

In certain embodiments, the first fusion protein and second fusion protein are incorporated into the tumor membrane vesicles by contacting the first fusion protein and second fusion protein with tumor membrane vesicles.

In certain embodiments, the first fusion protein, second fusion protein, and third fusion protein are incorporated into the tumor membrane vesicles by contacting first fusion protein, second fusion protein, and third fusion protein with tumor membrane vesicles.

In certain embodiments, the tumor membrane vesicles have an average diameter of about between 200 to 600 nanometers. In certain embodiments, the tumor membrane vesicles have an average diameter of about between 100 to 1000 nanometers. In certain embodiments, the tumor membrane vesicles have an average diameter of about between 50 to 10,000 nanometers.

In certain embodiments, the tumor antigen is human epidermal growth factor receptor 2 (HER2).

In certain embodiments, the dendritic cells are derived from bone marrow, peripheral blood, spleen, or other organ.

In certain embodiments, this disclosure relates to methods of treating cancer comprising administering an effective amount of dendritic cells pulsed in vitro with tumor membrane vesicles as disclosed herein to a subject in need thereof.

In certain embodiments, the tumor membrane vesicles are obtained from tumor tissue or cultured tumor cells from the subject to be treated. In certain embodiments, the tumor membrane vesicles are obtained from tumor tissue or cultured tumor cells not from the subject to be treated.

In certain embodiments, the cancer is breast cancer. In certain embodiments, the subject is diagnosed with a HER2 positive cancer.

In certain embodiments, the subject is diagnosed with triple negative breast cancer, i.e., tumor/cell sample is negative for estrogen receptor, negative for progesterone receptor, and negative for HER2.

In certain embodiments, the dendritic cells pulsed in vitro with tumor membrane vesicles are administered in combination with another chemotherapy agent.

### BRIEF DESCRIPTION OF THE SEVERAL VIEWS OF THE DRAWINGS

FIGS. 1A-1C show data on tumor membrane vesicles vaccine characterization and GPI-ISM incorporation.

FIG. 1A shows data on size distribution of TMVs (Zetasizer™) prepared from D2F2/E2 cells. The D2F2/E2 cell line were generated by transfecting D2F2 cells with full length human ErbB-2 (HER2).

FIG. 1B shows data on particle Z-Average (left panel) and polydispersity index (right panel) of four runs of D2F2/E2 TMVs.

FIG. 1C shows data on surface expression of HER2 antigen on D2F2/E2 cells (left panel) and TMVs prepared from D2F2/E2 cells (right panel).

FIGS. 2A-2C show data indicating TMV uptake by dendritic cells (DCs) is potentiated by GPI-ISMs.

FIG. 2A shows representative contour plots of bone marrow derived dendritic cells (BMDCs) after being in contact with TMVs. TMVs were labeled with the pH sensitive dye pHrodo Deep Red™ and incubated with BMDCs for 30, 90 or 180 min.

FIG. 2B shows data where BMDCs were analyzed by flow cytometry to quantify the percentage of cells that had taken up TMVs into the acidic endolyzosome which activates fluorescence of pHrodo™ labeled TMVs (n=3).

FIG. 2C shows data measurements of the pHrodo™ MFI of the BMDCs that had taken up TMVs (n=3).

FIGS. 3A-3E show data indicating TMVs with GPI-ISMs induce DC activation and cytokine release.

FIG. 3A shows representative plots of surface levels of CD86 and MHC-II on BMDCs after stimulating with LPS (positive control), D2F2/E2 TMVs containing different GPI-ISMs, or unstimulated (PBS) for 24 hrs.

FIG. 3B shows quantification as a percentage of CD86+ MHC-II+ BMDCs after 24-h pulsing with TMVs.

FIG. 3C shows ELISA data of IFN-γ levels in supernatants of BMDCs stimulated.

FIG. 3D shows ELISA data of TNF-α levels in supernatants of BMDCs stimulated.

FIG. 3E shows ELISA data of IL-6 levels in supernatants of BMDCs stimulated.

FIG. 4 shows data indicating DC vaccine inhibits D2F2/E2 tumor growth and increase the survival of mice. BALB/c mice (n=5 per group) were inoculated with 2×10⁵ D2F2/E2 cells s.c. on the flank. BMDCs were pulsed for 24 h with D2F2/E2 TMV (Plain TMV pulsed DC), or D2F2/E2 TMV incorporated with GPI-IL-12 and GPI-GM-CSF (DC vaccine) and administered at day 7 and 14 s.c. on the contralateral flank (1×10⁶ DCs/dose). Survival of tumor bearing mice is shown.

FIGS. 5A-5D show data indicating DC vaccine inhibits tumor growth, reduces lung metastasis, and increases immune cell infiltration into 4T1 tumors. The TNBC cell line 4T1 (CRL-2539) was obtained from ATCC.

FIG. 5A shows data where BALB/c mice (n=5 per group) were inoculated with 5×10⁴ 4T1 cells s.c. on the flank. DCs were pulsed for 24 h with 4T1 TMV (Plain TMV pulsed DC), or 4T1 TMV incorporated with GPI-IL-12 and GPI-GM-CSF (DC vaccine) and administered at days 5 and 12 s.c. on the contralateral flank at (1×10⁶ DCs/dose).

FIG. 5B shows data where lungs were harvested at day 20 and prepared into single cell suspension then grown in selection media containing 6-thioguanine to quantify metastatic 4T1 cells.

FIG. 5C shows quantification of MHC-II+ CD11c+ DCs from CD45+ cell in 4T1 tumors.

FIG. 5D shows quantification of CD4+ and CD8+ T cells from CD45+ cells in 4T1 tumors.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

An “embodiment” of this disclosure refers to an example, but not necessarily limited to such example. As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods.

“About” as used herein when referring to a measurable value such as an amount, a temporal duration, and the like, is meant to encompass variations of ±20% or ±10%, or ±5%, or ±1%, or ±0.1% from the specified value, as such variations are appropriate to perform the disclosed methods.

“Dendritic cells” refer to monocytes contained in bone marrow which also circulate in the bloodstream, and localized in tissues such as the skin, spleen, lung, and lymph nodes. In lymphoid organs, such as spleen and lymph nodes, dendritic cells present antigen in the context of an MHC-II-peptide complex to lymphocytes. Because cellular changes occur over time, there are various unique cell markers used to identify DCs in different maturation states, which can be evaluated in combination with the absence of other lineage markers, such as negative for CD3 (T cell), CD14 (monocyte), CD19 (B cell), CD56 (NK cell) and CD66b (granulocyte). In one example, classical human dendritic cells are reported to have the following markers: BDCA-1, CD8, CD8alpha, CD11b, CD11c, CD103, CD205, and MHC Class II. Pre-plasmacytoid DCs (pDCs) are reported to have the following markers: BDCA-2, BDCA-4, CD11c(low), CD45RA, CD123, ILT-7, MHC Class II(low), TLR7, and TLR9. See Merad et al. The Dendritic Cell Lineage: Ontogeny and Function of Dendritic Cells and Their Subsets in the Steady State and the Inflamed Setting, Annu Rev Immunol, 2013, 31: 563-604 and Schraml et al. Defining dendritic cells, 2015, Curr Opin Immunol, 32:13-20. One method of isolating dendritic cells is by isolating bone marrow cells and culturing the dendritic cells from the bone marrow cells in a growth medium containing exogenously added GM-CSF, IL-4, and TNF. See for example, Kelly Roney, Bone Marrow-Derived Dendritic Cells, Chapter 4, 2019.

As used herein, “subject” refers any animal, preferably a human patient, livestock, or domestic pet.

As used herein, the terms “treat” and “treating” are not limited to the case where the subject (e.g., human patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

“Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others.

The cancer to be treated in the context of the present disclosure may be any type of cancer or tumor such as lung cancer, non-small cell lung cancer and subtypes of NSCLC such as adenocarcinoma, squamous cell carcinoma, and large cell carcinoma, and small cell lung cancer. Contemplated are malignancies located in the colon, abdomen, bone, breast, digestive system, liver, pancreas, peritoneum, endocrine glands (adrenal, parathyroid, hypophysis, testicles, ovaries, thymus, thyroid), eye, head and neck, nervous system (central and peripheral), lymphatic system, pelvis, skin, soft tissue, spleen, thorax and genito-urinary apparatus and, more particularly, adrenocortical carcinoma, AIDS-related lymphoma, AIDS-related malignant tumors, anal cancer, astrocytoma, cancer of the biliary tract, cancer of the bladder, bone cancer, brain stem glioma, brain tumors, breast cancer, cancer of the renal pelvis and ureter, primary central nervous system cerebellar astrocytoma, brain astrocytoma, cancer of the cervix, chronic lymphocytic leukemia, chronic myeloid leukemia, cancer of the colon, cutaneous T-cell lymphoma, endocrine pancreatic islet cells carcinoma, endometrial cancer, ependymoma, epithelial cancer, cancer of the esophagus, Ewing's sarcoma and related tumors, cancer of the exocrine pancreas, extracranial germ cell tumor, extragonadal germ cell tumor, extrahepatic biliary tract cancer, cancer of the eye, Gaucher's disease, cancer of the gallbladder, gastric cancer, gastrointestinal carcinoid tumor, gastrointestinal tumors, germ cell tumors, gestational trophoblastic tumor, head and neck cancer, hepatocellular cancer, hypergammaglobulinemia, hypopharyngeal cancer, Hodgkin's disease, intestinal cancers, intraocular melanoma, islet cell carcinoma, islet cell pancreatic cancer, Kaposi's sarcoma, cancer of the larynx, cancer of the lip and mouth, macroglobulinemia, malignant mesothelioma, malignant thymoma, medulloblastoma, melanoma, mesothelioma, occult primary metastatic squamous neck cancer, primary metastatic squamous neck cancer, metastatic squamous neck cancer, multiple myeloma, multiple myeloma/plasmatic cell neoplasia, myelodysplastic syndrome, myelogenous leukemia, myeloid leukemia, myeloproliferative disorders, paranasal sinus and nasal cavity cancer, nasopharyngeal cancer, neuroblastoma, non-Hodgkin's lymphoma, non-melanoma skin cancer, metastatic squamous neck cancer with occult primary, buccopharyngeal cancer, malignant fibrous histiocytoma, malignant fibrous osteosarcoma/histiocytoma of the bone, epithelial ovarian cancer, ovarian germ cell tumor, ovarian low malignant potential tumor, pancreatic cancer, paraproteinemias, purpura, parathyroid cancer, cancer of the penis, hypophysis tumor, neoplasia of plasmatic cells/multiple myeloma, primary central nervous system lymphoma, primary liver cancer, prostate cancer, rectal cancer, renal cell cancer, cancer of the renal pelvis and ureter, retinoblastoma, rhabdomyosarcoma, cancer of the salivary glands, sarcoidosis, sarcomas, skin cancer, small cell lung cancer, small intestine cancer, soft tissue sarcoma, squamous neck cancer, stomach cancer, pineal and supratentorial primitive neuroectodermal tumors, T-cell lymphoma, testicular cancer, thymoma, thyroid cancer, transitional cell cancer of the renal pelvis and ureter, transitional renal pelvis and ureter cancer, trophoblastic tumors, cell cancer of the renal pelvis and ureter, cancer of the urethra, cancer of the uterus, uterine sarcoma, vaginal cancer, optic pathway and hypothalamic glioma, cancer of the vulva, Waldenstrom's macroglobulinemia, Wilms' tumor and any other hyperproliferative disease, as well as neoplasia, located in the system of a previously mentioned organ.

In certain embodiments, compounds disclosed herein may be administered in combination with an additional anti-cancer agent. A “chemotherapy agent,” “chemotherapeutic,” “anti-cancer agent” or the like, refer to molecules that are recognized to aid in the treatment of a cancer. Contemplated examples include the following molecules or derivatives such as abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, ribociclib, tisagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof such as cyclophosphamide, methotrexate, 5-fluorouracil (CMF); doxorubicin, cyclophosphamide (AC); mustine, vincristine, procarbazine, prednisolone (MOPP); adriamycin, bleomycin, vinblastine, dacarbazine (ABVD); cyclophosphamide, doxorubicin, vincristine, prednisolone (CHOP); rituximab, cyclophosphamide, doxorubicin, vincristine, prednisolone (RCHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC). In certain embodiments, the chemotherapy agent is an antibody, anti-PD-1, anti-PD-L1, anti-CTLA4 antibody or combinations thereof, such as an anti-CTLA4 (e.g., ipilimumab, tremelimumab), an anti-PD-L1 (e.g., atezolizumab, avelumab, durvalumab) or an anti-PD1 antibody (e.g., nivolumab, pembrolizumab, cemiplimab, dostarlimab, spartalizumab, camrelizumab, tislelizumab, toripalimab, sintilimab).

The term “nucleic acid” refers to a polymer of nucleotides, or a polynucleotide, e.g., RNA, DNA, or a combination thereof. The term is used to designate a single molecule, or a collection of molecules. Nucleic acids may be single stranded or double stranded and may include coding regions and regions of various control elements.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

A “heterologous” nucleic acid sequence or peptide sequence refers to a nucleic acid sequence or a peptide sequence that does not naturally occur, e.g., because the whole sequence contains a segment from other plants, bacteria, viruses, other organisms, or joinder of two sequences that occur the same organism but are joined together in a manner that does not naturally occur in the same organism or any natural state.

The term “recombinant” when made in reference to a nucleic acid molecule refers to a nucleic acid molecule which is comprised of segments of nucleic acid joined together by means of molecular biological techniques provided that the entire nucleic acid sequence does not occurring in nature, i.e., there is at least one mutation in the overall sequence such that the entire sequence is not naturally occurring even though separately segments may occur in nature. The segments may be joined in an altered arrangement such that the entire nucleic acid sequence from start to finish does not naturally occur. The term “recombinant” when made in reference to a protein or a peptide refers to a protein molecule that is expressed using a recombinant nucleic acid molecule.

The terms “vector” or “expression vector” refer to a recombinant nucleic acid containing a desired coding sequence and appropriate nucleic acid sequences necessary for the expression of the operably linked coding sequence in a particular host organism or expression system, e.g., cellular or cell-free expression systems. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals. In certain embodiments, this disclosure contemplates a vector encoding a peptide disclosed herein in operable combination with a heterologous promoter.

Protein “expression systems” refer to in vivo and in vitro (cell free) systems. Systems for recombinant protein expression typically utilize somatic cells transfected with a DNA expression vector that contains the template. The cells are cultured under conditions such that they translate the desired protein. Expressed proteins are extracted for subsequent purification. In vivo protein expression systems using prokaryotic and eukaryotic cells are well known. Proteins may be recovered using denaturants and protein-refolding procedures. In vitro (cell-free) protein expression systems typically use translation-compatible extracts of whole cells or compositions that contain components sufficient for transcription, translation, and optionally post-translational modifications such as RNA polymerase, regulatory protein factors, transcription factors, ribosomes, tRNA cofactors, amino acids, and nucleotides. In the presence of an expression vectors, these extracts and components can synthesize proteins of interest. Cell-free systems typically do not contain proteases and enable labeling of the protein with modified amino acids. See, e.g., Shimizu et al., Cell-free translation reconstituted with purified components, 2001, Nat. Biotechnol., 19, 751-755 and Asahara & Chong, Nucleic Acids Research, 2010, 38(13): e141, both hereby incorporated by reference in their entirety.

### Tumor Membrane Vesicles (TMVs)

In certain embodiments, the tumor membrane vesicles (TMVs) contemplated to be pulsed with dendritic cells include those as described in PCT patent application PCT/US2013/024355 (WO2013/116656), the contents of which are herein incorporated by reference in its entirety.

In certain embodiments, the TMV is a particle formed from cell or cell membrane material obtained from a tumor cell or tissue (e.g., surgically resected patient tumor tissue). Because the TMV contains tumor cell membrane material, the TMV contains tumor associated molecules and/or tumor-specific molecules (e.g., antigens). These tumor-specific antigens can activate the subject's immune system by active immunization with tumor antigens. Thus, TMVs represent a personalized, tissue-derived strategy for treating tumors in a subject.

In certain embodiments, the TMV contains a lipid membrane comprised of tumor associated molecules and/or tumor specific molecules (e.g., antigens). Further, additional molecules not specifically derived from a tumor or tumor sample can be attached to the lipid membrane. These additional molecules include one or more immunostimulatory agents, one or more antigens, and one or more additional anti-tumor compounds (e.g., anti-cancer agent). The lipid membrane may be in the form of a monolayer or bilayer (e.g., a phospholipid monolayer or phospholipid bilayer), or mixtures thereof.

In certain embodiments, the TMV contains an immunostimulatory agent (ISM) attached to the lipid membrane of the TMV. As used herein, an “immunostimulatory agent” is any molecule that, when attached to a TMV, can stimulate, or co-stimulate an anti-tumor immune response. TMVs containing membrane-attached immunostimulatory agents deliver molecules which stimulate immune responses, as well as patient-specific tumor antigens, and activate immune cells to promote an anti-tumor immune response.

In certain embodiments, the immunostimulatory agent is IL-12, GM-CSF, IL-2 or combinations thereof. In certain embodiments, the immunostimulatory agent is IL-12, GM-CSF, and B7-1 (also known as CD80), B7-2. In certain embodiments, immunostimulatory agent is IL-12, GM-CSF, and B7-2 (also known as CD86). In certain embodiments, the immunostimulatory agent is B7-1, B7-2, IL-12, or combinations thereof. In certain embodiments, the immunostimulatory agent is B7-1, IL-12, or combinations thereof. In some embodiments, the TMV includes one immunostimulatory agent or, alternatively, two or more immunostimulatory agents.

Incorporating Glycosyl Phosphatidylinositol (GPI)-Anchored Immunostimulatory Molecules (GPI-ISMs) onto the Surface of TMVs

A number of proteins commonly expressed by cells are attached to the cell membrane via a GPI-anchor. These proteins are post-translationally modified at their carboxy terminus to express this glycosylated moiety which is synthesized in the endoplasmic reticulum. These naturally expressing GPI-anchored molecules are widely distributed in mammalian cells and serve a host of different cellular functions, such as cell adhesion, enzymatic activity, and complement cascade regulation. The GPI-anchor consists of a glycosylated moiety attached to phosphatidylinositol containing fatty acids. The phosphatidylinositol portion, as well as an ethanolamine which is attached to the C-terminal of the extracellular domain of the membrane proteins, anchor the molecule to the cell membrane lipid bilayer.

In order to exploit this natural linkage using recombinant DNA techniques, the transmembrane and cytoplasmic domains of a transmembrane surface protein need only be replaced by the signal sequence for GPI-anchor attachment that is found at the hydrophobic C-terminus of GPI-anchored protein precursors. This method may be used to generate GPI-anchored proteins is not limited to membrane proteins. Attaching a GPI-anchor signal sequence to a secretory protein also converts the secretory protein to a GPI-anchored form. The method of incorporating the GPI-anchored proteins onto isolated cell surfaces or TMVs is referred to here as protein transfer.

GPI-anchored molecules can be incorporated onto lipid membranes spontaneously. GPI-anchored proteins can be purified from one cell type and incorporated onto cell membranes of a different cell type. GPI-anchored proteins can be used to customize the lipid membranes disclosed herein. Multiple GPI-anchored molecules can be simultaneously incorporated onto the same cell membrane or TMV. The amount of protein attached to the TMV can be controlled by simply varying the concentration of the GPI-anchored molecules to be incorporated onto membranes. These features make the protein transfer approach a more viable choice for the development of cancer vaccines for clinical settings. The molecules incorporated by means of protein transfer retain their functions associated with the extracellular domain of the native protein. Cells and isolated membranes can be modified to express immunostimulatory agents. In certain embodiments, the disclosure contemplates that the GPI-anchored molecules are incorporated onto the surface of TMVs by this protein transfer method. GPI-anchored proteins attached to the surface of TMVs are used for an array of functions including immunostimulation, co-stimulation, boosting immune responses, generating long term memory, etc., thereby enhancing the capacity to function as a targeted therapy for cancer treatment.

Dendritic Cells Pulsed with Cytokine-Adjuvanted Tumor Membrane Vesicles and Uses in Cancer Treatments

A tumor membrane vesicle (TMV)-based vaccine immunotherapy approach was developed that can be prepared from frozen tumor tissue. The TMVs used were 200-600 nm in size, making them ideal for processing by antigen presenting cells, such as dendritic cells (DCs) and macrophages. To increase the immune response against the tumor antigens in the TMV, a “protein transfer” approach was developed that incorporates glycosyl phosphatidylinositol (GPI)-anchored immunostimulatory molecules (GPI-ISMs) onto the surface of TMVs.

TMVs incorporated with GPI-B7-1 and GPI-IL-12 were utilized to stimulate the anti-tumor response in preclinical models of breast cancer, thymoma, and head-and-neck cancers. While these approaches are promising when the tumor tissue is available in sufficient quantity, it is difficult to generate sufficient TMV vaccine for the cancers where the tissue is limited in quantity, such as inoperable tumors or metastatic recurrent cases where the archived primary tumor is no longer available at the time of recurrence where only a biopsy sample can be used. To overcome this issue, once can provide a dendritic cell (DC vaccine) by delivering a dose of TMVs to antigen presenting cells ex vivo enhancing the TMV uptake, DC activation, and anti-tumor immune response in a therapeutic setting.

A DC vaccine platform was developed that utilizes autologous DCs pulsed with cytokine-adjuvanted TMVs as a therapeutic in murine breast cancer models. DCs can take up TMVs when pulsed in vitro. Uptake is improved when TMVs contain GPI-GM-CSF and GPI-IL-12 on their surface. TMVs containing GPI-GM-CSF and GPI-IL-12 can also improve DC activation as measured by surface CD86 and MHC-II expression as well as cytokine production. Treatment with DCs pulsed ex vivo with HER-2+ TMV results in tumor growth inhibition and improved survival of tumor bearing mice. Further, in a triple negative breast cancer model which lacks a defined target antigen, the DC vaccine can also inhibit tumor growth. Experiments also indicate that DCs pulsed with TMVs can reduce metastasis and increase immune cell infiltration into tumors.

Experiments indicates that a DC vaccine inhibited tumor growth in two different breast cancer models. DCs were presented simultaneously with tumor antigens and adjuvants (GPI-ISMS) in a particulate form to enhance uptake of the vaccine and activation of DCs. DCs pulsed with TMVs had improved uptake. GPI-GM-CSF on TMVs induced DC activation. GPI-GM-CSF and GPI-IL-12 on TMVs provided signaling to produce inflammatory cytokines. The DC vaccine platform was tested in the metastatic 4T1 model (triple negative breast cancer cells), which lacks a defined tumor antigen, where vaccine pulsed DCs resulted in the inhibition of tumor growth. Reductions of lung metastatic burden and increased tumor infiltrating immune cells were also ob served.

Particles that are within the 100 nm to 1 μm diameter have immunogenicity and uptake by DCs. TMVs are derived from plasma membrane of tumor cells and have an average diameter of 200 nm, falling within this range, and can retain the surface antigens that are present on tumor cells.

This disclosure relates to methods of treating cancer using dendritic cells pulsed with tumor membrane vesicles comprising tumor antigens and cytokines. In certain embodiments, tumor membrane vesicles contain fusion proteins with a cytokine surface protein linked to glycosyl phosphatidylinositol domain anchoring the fusion protein to the tumor membrane; thus, resulting in dendritic cells that contain cytokine surface protein(s). In certain embodiments, the cytokine is granulocyte-macrophage colony-stimulating factor (GM-CSF) and optionally other immunostimulatory agents such as interleukins, e.g., interleukin 12.

In certain embodiments, the dendritic cells are derived from bone marrow, spleen, lymphoid tissue, or peripheral blood.

In certain embodiments, the immunostimulatory agent or cytokine is selected from IFN-gamma, TNF-alpha, IL-2, IL-12, IL-18, IL-22, IL-23, and combinations thereof.

In certain embodiments, tumor membrane vesicles or pulsed dendritic cells comprise GM-CSF, an immunostimulatory agent or cytokine, e.g., IL-12, and optionally B7-1 and/or B7-2 anchored to the tumor membrane or dendric cell membrane.

In certain embodiments, this disclosure relates to dendritic cells pulsed in vitro with tumor membrane vesicles comprising: a tumor cell antigen on the surface of the tumor membrane vesicles; a first fusion protein on the surface of the tumor membrane vesicles having a granulocyte-macrophage colony-stimulating factor (GM-C SF) anchoring the first fusion protein to the tumor membrane and a glycosyl phosphatidylinositol domain; and second fusion protein on the surface of the tumor membrane vesicles having interleukin 12 and a glycosyl phosphatidylinositol domain anchoring the first fusion protein to the tumor membrane.

In certain embodiments, this disclosure relates to dendritic cells pulsed or contacted in vitro with tumor membrane vesicles comprising: a tumor cell antigen on the surface of the tumor membrane vesicles; a first fusion protein on the surface of the tumor membrane vesicles having a granulocyte-macrophage colony-stimulating factor (GM-CSF) and a glycosyl phosphatidylinositol domain; and a second fusion protein on the surface of the tumor membrane vesicles having interleukin 12 and a glycosyl phosphatidylinositol domain.

In certain embodiments, the tumor membrane vesicles (TMV) are obtained from a tumor tissue or cultured tumor cells. In certain embodiments, the tumor membrane vesicles (TMV) contain a third fusion protein comprising a cancer/tumor antigen and/or immunostimulatory molecule, and a glycosyl phosphatidylinositol domain.

In certain embodiments, the first fusion protein and second fusion protein are incorporated into the tumor membrane vesicles by contacting the first fusion protein and second fusion protein with the tumor membrane vesicle. In certain embodiments, the first fusion protein and second fusion protein are incorporated into the tumor membrane vesicles by contacting the first fusion protein and second fusion protein with a tumor cell that produces tumor membrane vesicle or plasma membrane structures extracted from a parental tumor cell providing nano-sized tumor membrane vesicles.

In certain embodiments, the first fusion protein, second fusion protein, and third fusion protein are incorporated into the tumor membrane vesicles by contacting first fusion protein, second fusion protein, and optionally a third fusion protein, e.g., comprising a cancer antigen and/or other immunostimulatory molecule, with the tumor membrane vesicle.

In certain embodiments, the cancer antigen is human epidermal growth factor receptor 2 (HER2). In certain embodiments, the cancer antigen is selected from human epidermal growth factor receptor 2 (HER-2), prostate-specific antigen (PSA), prostatic acid phosphatase (PAP), (EGFR) epidermal growth factor receptor, (MUC1) mucin1, (MUC16) mucin16, (EpCAM) epithelial cell adhesion molecule, (AFP) alpha-fetoprotein, (FAP) familial adenomatous polyposis, (CEA) carcinoembryonic antigen, (PSCA) prostate stem cell antigen, (PSMA) prostate-specific membrane antigen, (AXL) AXL receptor tyrosine kinase, (DLL3) delta-like 3, (EPHA2) EPH receptor A2, (FRα) folate receptor alpha, (LMP1) Epstein-Barr virus latent membrane protein 1, (MAGE) melanoma antigen gene protein, MAGE-A1, MAGE-A3, MAGE-A4, (DR5) death receptor 5, (NKG2D) natural killer group 2 member D receptor, (CAIX) carbonic anhydrase IX, (TAG-72) tumor-associated glycoprotein 72, (GUCY2C) guanylate cyclase 2C, (ANTXR1) anthrax toxin receptor 1, (GSPG4) general secretion pathway protein G, (ROR) RAR-related orphan receptors, IL13RA2 (Interleukin 13 Receptor Subunit Alpha 2), Wilms' tumor 1 (WT1), Survivin, Tn (aGalNAc-O-Ser/Thr), sialyl-Tn (aNeuAc2,6-aGalNAc-O-Ser/Thr), TF (bGal1,3-aGalNAc-O-Ser/Thr), CA 19-9 (Neu5Acα2-3Galβ1-3[Fucα1-4]GlcNAcβ), Telomerase reverse transcriptase (TERT), Beta-hCG (Human chorionic gonadotropin), p53, Ras, bladder tumor antigen (BTA), antibody specific antigen Om5, GD2 (Ganglioside GD2), integrin alpha-v/beta-6, or mesothelin antigen.

In certain embodiments, the antigen molecule may be anchored onto the membrane of TMVs or dendritic cells through a variety of linkages, such as via lipid palmatic acid, biotin-avidin interaction, or a glycosylphosphatidylinositol (GPI)-anchor.

In certain embodiments, the tumor membrane vesicles have a diameter of about 200 to 600 nanometers. In certain embodiments, the tumor membrane vesicles have a diameter of about 100 to 1000 nanometers. In certain embodiments, the tumor membrane vesicles have a diameter of about 50 to 10,000 nanometers.

In certain embodiments, the tumor membrane vesicles may be extracted from cells or parental tumor cells.

In certain embodiments, the cells or parental tumor cells are harvested and resuspended in a hypotonic buffer and subjected to fragmentation by external forces.

In certain embodiments, the cells or parental tumor cells are exposes to a cycle of freezing and thawing cycles.

In certain embodiments, the cells or parental tumor cells are subjected to Dounce homogenization using pestles (known as the “loose” and “tight” pestles), which have a specified outer diameter, relative to the inner diameter of the cylinder. For example, a loose pestle may have a clearance from the cylinder wall of about 0.0025-0.0055 inches while the tight pestle may have a clearance of about 0.0005-0.0025 inches. The cells are lysed by shear stress with minimal (if any) degree of heating, thereby leaving enzyme complexes largely intact.

In certain embodiments, the tumor membrane vesicles are separated from other cellular components by centrifugation (e.g., continuous high-speed or density gradient centrifugation). In certain embodiments, the centrifugation is at 1000 g, 10000 g, and 100000 g. In certain embodiments, nuclei, organelles, and other impurities are remove from the TMVs.

In certain embodiments, the tumor cell membranes are separated by density gradient centrifugation. In certain embodiments, the cell membranes are separated by centrifugation through discontinuous sucrose (w/v) density gradient, e.g., 30-40-55%.

In certain embodiments, the cell membranes are isolated at the interface of the different sucrose solutions. In certain embodiments, the lipid rings may be detected and collected. In certain embodiments, fractions between 30% and 40% sucrose are collected.

In certain embodiments, the tumor membrane vesicles are sonicated and extruded through membranes (e.g., polycarbonate membranes) to obtain desired sizes of tumor membrane vesicles.

In certain embodiments, the tumor membranes vesicles comprise cytoplastic proteins and RNAs.

In certain embodiments, this disclosure relates to methods of treating cancer comprising administering an effective amount of dendritic cell pulsed in vitro with tumor membrane vesicles as disclosed herein to a subject in need thereof.

In certain embodiments, the tumor membrane vesicles are obtained from tumor tissue or cultured tumor cells from the subject to be treated. In certain embodiments, the tumor membrane vesicles are obtained from tumor tissue or cultured tumor cells not from the subject to be treated.

In certain embodiments, the cancer is breast cancer. In certain embodiments, the subject is diagnosed with a HER2 positive cancer. In certain embodiments, the subject is diagnosed with triple negative breast cancer. In certain embodiments, the triple negative breast cancer is a metastatic triple negative breast cancer.

In certain embodiments, the cancer is lung cancer. In certain embodiments, the subject is diagnosed with metastatic lung cancer.

In certain embodiments, the dendritic cell pulsed in vitro with tumor membrane vesicles are administered in combination with another chemotherapy agent.

In certain embodiments, the chemotherapy agent is an anti-CTLA4 antibody, an anti-PD1 antibody, and an anti-PD-L1 antibody.

In certain embodiments, treatment reduces metastasis of the triple negative breast cancer.

In certain embodiments, treatment reduces the size of a tumor.

In certain embodiments, methods comprise administering to the subject a therapeutically effective amount of an immunotherapeutic agent. As such, a combination therapy comprising dendritic cells pulsed with TMVs as disclosed herein and an immunotherapeutic agent is administered. Administering a combination of an immunotherapeutic agent with dendritic cells pulsed with TMV and immunotherapy can significantly enhance immune responses and increases response rates. In certain embodiments, one is able to induce anti-tumor immunity and infiltration of immune cells into TNBC tumor tissue, which is a positive prognostic indicator. See van Rooij en et. al., Pharmacol. Ther., 156:90-101 (2015).

In certain embodiments, methods comprise using dendritic cells pulsed with TMV as disclosed herein and an immunotherapeutic agent in a combination therapy that generates protective immunity, reduces metastasis, and prolongs survival. Additionally, the inclusion of an exogenous antigen molecule in the TMV of the pulsed dendric cell and immunotherapeutic agent combination therapy can aid in disrupting metastasis.

In certain embodiments, the immunotherapeutic agent is an immune checkpoint inhibitor (ICI). In certain embodiments, the immunotherapeutic agent is an antibody, particularly an antibody having ICI function. In certain embodiments, the immunotherapeutic agent can include one or more of an anti-CTLA4 antibody, an anti-PD1 antibody, an anti-PDL1 antibody, or any combination thereof.

In certain embodiments, the anti-CTLA4 antibody is abatacept, belatacept, ipilimumab, tremelimumab, or any combination thereof. In certain embodiments, the anti-CTLA4 antibody is ipilimumab. In certain embodiments, the anti-PD-L1 antibody can include atezolizumab, durvalumab, avelumab, or any combination thereof. In certain embodiments, the anti-PDL1 antibody is atezolizumab, durvalumab (MEDI4736), avelumab, or any combination thereof. In certain embodiments, the PD-1 inhibitor can include, for example, nivolumab, pembrolizumab, pidilizumab, AMP-244 (Amplimmune/GSK), BMS-936559 (BMS), and MEDI4736 (Roche/Genentech). In certain embodiments, the anti-PD1 antibody is nivolumab, pembrolizumab, or any combination thereof. In certain embodiments, the administering step can include substitution of an anti-cancer agent for the immunotherapeutic agent. In certain embodiments, the administering step can include administering the immunotherapeutic agent in combination with another anti-cancer agent. The anti-cancer agent can be any herein disclosed anti-cancer agent.

In certain embodiments, the method further comprises administering an adjuvant. The adjuvant can be administered prior to, concurrent with, or subsequent to administration of the dendritic cell pulsed with TMV as disclosed herein and the immunotherapeutic agent. In some embodiments, the adjuvant is GM-CSF, or any biocompatible FDA-approved adjuvant. In some embodiments, the adjuvant comprises alum, IL-2, ICAM-1, GM-CSF, flagellin, unmethylated, CpG oligonucleotide, lipopolysaccharides, lipid A or Quillaja saponin-based adjuvants (QS-21) derived from the soap bark tree (Quillaja saponaria). The adjuvant can be in a form separate from the dendritic cells pulsed with TMVs or can be anchored to the lipid membrane of the TMV (by, for example, via a GPI anchor). In some embodiments, the dendritic cells pulsed with TMVs further comprises an adjuvant anchored to the lipid membrane wherein the adjuvant and antigen molecule are not the same molecule.

In certain embodiments, the administering step can include any method of introducing the dendritic cells pulsed with TMV as disclosed herein optionally in combination with another anticancer agent into the subject. The administering step can include at least one, two, three, four, five, six, seven, eight, nine, or at least ten dosages. In certain embodiments, the administering step can be performed before a subject exhibits any disease symptoms (e.g., prophylactically), or during or after disease symptoms occur or after other treatment modalities such as surgery, chemotherapy, and radiation. In certain embodiments, the administering step can be performed prior to, concurrent with, or subsequent to administration of other agents to the subject. In certain embodiments, administering step can be performed with or without co-administration of additional agents (e.g., additional immunostimulatory agents, anti-cancer agents).

In certain embodiments, methods can include systemic administration (e.g., injection into the circulatory or lymphatic systems) of the dendritic cells pulsed with TMV as disclosed herein. In certain embodiments, the method can include local administration of the dendritic cells pulsed with TMVs as disclosed herein. For example, an anticancer agent can be administered locally to a tumor or an area near a tumor. In some embodiments, the dendritic cells pulsed with TMVs as disclosed herein are administered to areas of the subject comprising tumors. Alternatively, the method can include systemic administration of an anticancer agent and local administration of dendritic cells pulsed with TMVs as disclosed herein.

In certain embodiments, the treatment comprising administering to a subject a therapeutically effective amount of dendritic cells pulsed with TMVs as disclosed herein reduces metastasis of triple negative breast cancer. In some embodiments, the treatment reduces the size of a tumor. In some embodiments, the treatment does not result in substantial liver toxicity.

In certain embodiments, the cancer is lung cancer. In certain embodiments, the subject is diagnosed with a metastatic cancer. In certain embodiments, the subject is diagnosed with metastatic lung cancer.

In certain embodiments, this disclosure relates to the production of a medicament comprising dendritic cells pulsed with TMVs as disclosed herein for use in treating cancer in a subject as disclosed herein. In certain embodiments, the subject is a human subject.

### TMV Preparation, Characterization, and GPI-ISM Incorporation

TMVs were generated from frozen tumor tissue or cultured cell pellets. Tumor tissue was homogenized, and the plasma membranes were isolated by ultracentrifugation over a 41% sucrose gradient. TMVs were analyzed for particle diameters using a Zetasizer™. The GPI-ISMs GPI-IL-12 and GPI-GM-CSF were incorporated using protein transfer by incubating 25 μg of purified GPI-ISMs per milligram of TMV at 37 C for 4 h under constant rotation. TMVs with incorporated GPI-ISMs were then pelleted by ultracentrifugation and unbound GPI-ISMs in the supernatant were removed, followed by a wash with PBS. Incorporation of GPI-GM-CSF and GPI-IL-12 was verified via flow cytometry by using anti-mouse GM-CSF PE (clone MP1-22E9) and anti-mouse IL-12 APC (clone C17.8) antibodies.

### TMVs Containing the Tumor Antigen HER2 and Protein Transferred GPI-ISMs on Their Surface

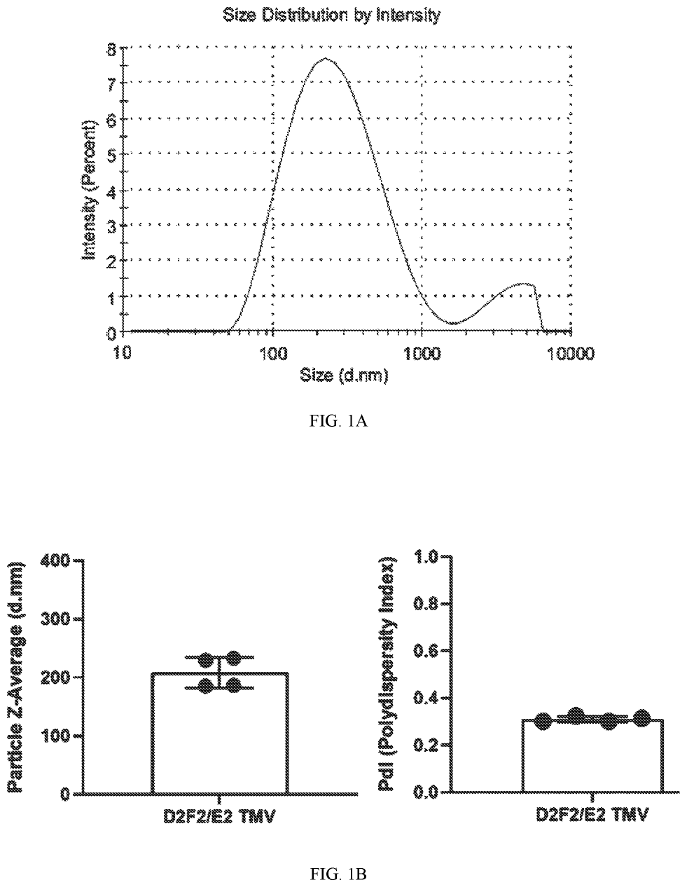

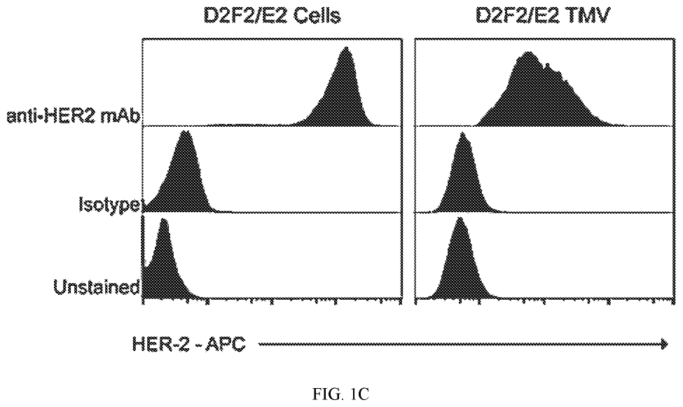

To characterize TMVs, flow cytometry analysis was used to assess the surface marker expression and size distribution of the vesicles (Zetasizer™). The data suggest that D2F2/E2 TMVs prepared from cell culture pellets are within 200 nm in diameter (FIGS. 1A,B). The polydispersity index of the vesicles shows an average measurement of 0.31, which is similar to that of liposome formulations. Surface marker expression of HER2 was performed on D2F2/E2 cells and TMVs from this cell line. D2F2/E2 cells express high levels of HER2 on the surface, and TMVs retain this tumor antigen on their surface (FIG. 1C). Flow cytometry analysis of plain TMVs show no signal of IL-12 or GM-CSF on their surface, while analysis of GPI-ISM-incorporated TMV (TMV vaccine) confirms the presence of protein transfer-mediated incorporation of GPI-GM-CSF and GPI-IL-12.

### Uptake of TMVs by DCs is Potentiated by GPI-ISMs

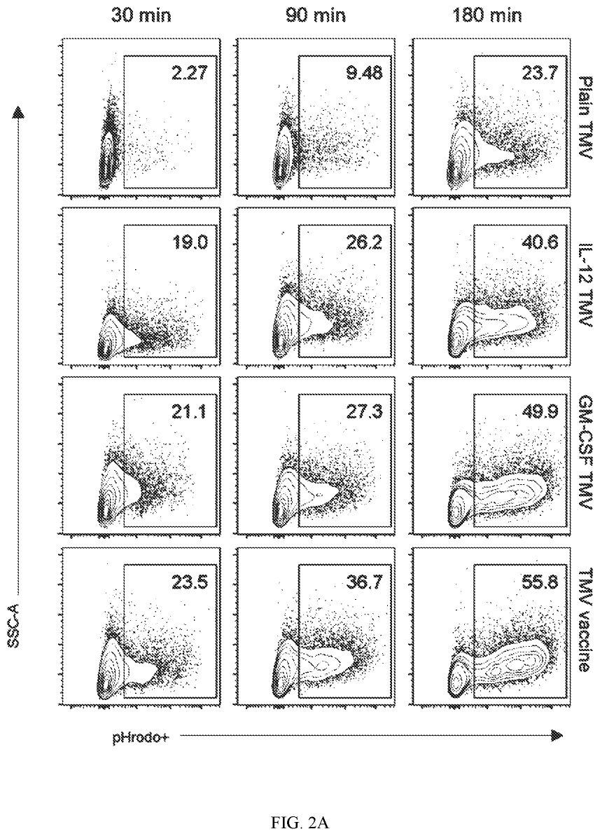

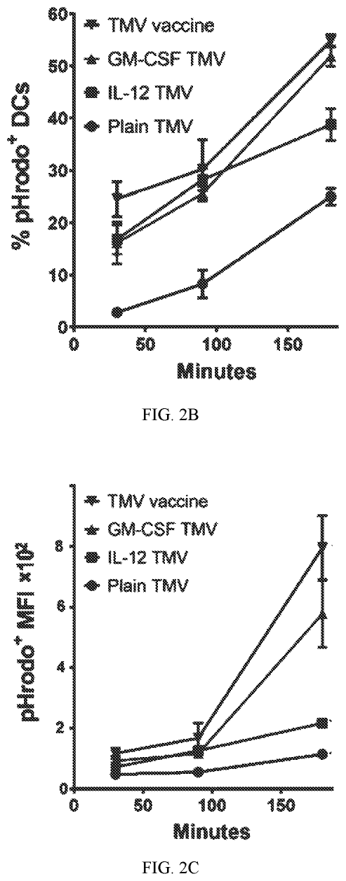

DCs are one of the most potent APCs that can take up vaccine antigens at the vaccination site and migrate to lymphoid organs to initiate a T cell response. To test whether DCs can phagocytose TMVs incorporated with GPI-ISMs, TMVs were labeled with the pH sensitive dye pHrodo Deep Red™, which can fluoresce in the acidic conditions of the late endosome/phagolysosome as described in methods. BMDCs were pulsed with pHrodo™ labeled TMVs for 30, 90, and 180 min, then washed and kept on ice until flow cytometry analysis. DCs that were pulsed with plain TMV were able to take up TMVs as early as 30 min, albeit at a smaller percentage compared to DCs pulsed with TMV containing either GPI-ISM. After 180 min of incubation, there is a significant increase in the percentage of pHrodo™+ DCs pulsed with GPI-IL-12 TMVs; however, the highest percentages of pHrodo™+ DCs were observed after pulsing with GPI-GM-CSF TMVs or TMV vaccine containing both GPI-ISMs (FIGS. 2A and 2B). There is also a significant increase in the pHrodo™ MFI on DCs pulsed with TMVs containing either GPI-IL-12 or GM-CSF compared to plain TMVs; however, DCs pulsed with TMVs containing both GPI-IL-12 and GPI-GM-CSF showed the highest MFI (FIG. 2C).

TMVs Incorporated with GPI-ISMs Can Induce DC Activation and Cytokine Production

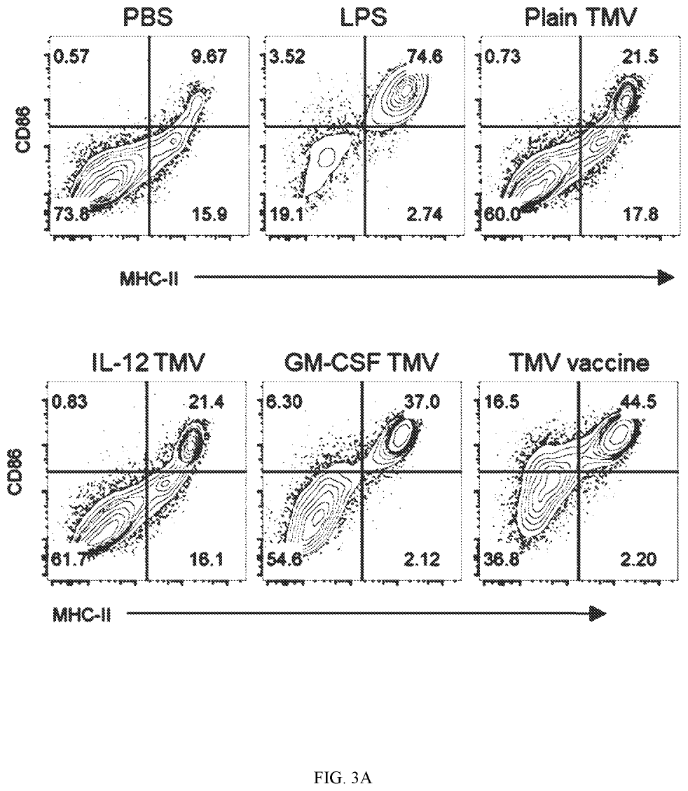

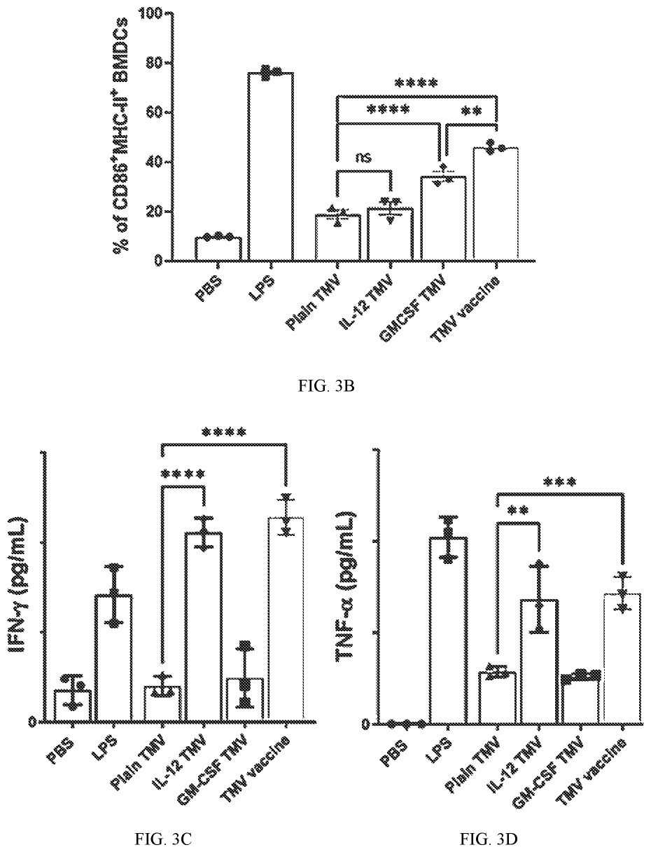

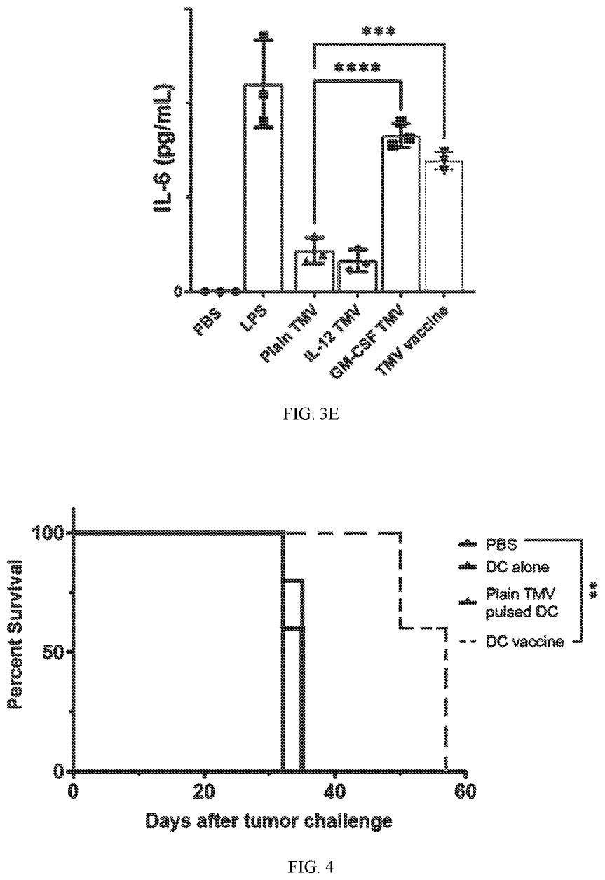

Given that DCs were able to take up TMVs in vitro, the ability of these cells to upregulate the activation markers CD86 and MHC-II were tested along with production of inflammatory cytokines after pulsing with TMVs made from D2F2/E2 cells. DCs pulsed with plain TMVs or TMVs containing GPI-IL-12 showed no significant activation as measured by CD86 and MHC-II expression (FIGS. 3A,B). DCs that were pulsed with TMVs containing GPI-GM-CSF or both GPI-ISMs showed a significant increase in activation marker expression (FIG. 3B). Cytokine production was measured as a proxy to GPI-ISM functionality. DCs pulsed with TMVs containing GPI-IL-12 showed a slight increase in production of IFN-compared to plain TMV pulsing (FIG. 3C). Similarly, those DCs that were pulsed with GPI-IL-12 TMVs also showed an increase in TNF-production (FIG. 3D). Those DCs that were pulsed with GPI-GM-CSF TMVs showed an increased production of IL-6 (FIG. 3E) but no increase in IFN- or TNF-. The presence of GPI-GM-CSF did not inhibit or potentiate the production of cytokines induced in the presence of GPI-IL-12, as evidenced by the cytokine production of DCs pulsed with the TMV vaccine containing both GPI-ISMs.

### DC Vaccine Therapy Reduces Tumor Growth

To test the efficacy of the DCs pulsed with TMVs as a vaccine platform, the HER2 positive murine model D2F2/E2 was used. The D2F2/E2 model represents an immunogenic tumor model due to the presence of the human tumor antigen HER2. Mice were challenged with D2F2/E2 cells and BMDCs were delivered s.c. on the opposite flank after seven days of tumor growth. Treatment with DCs that were not pulsed with TMV (DC alone), and DCs that were pulsed with TMVs without GPI-ISMs (plain TMV pulsed DCs) showed no reduction of tumor growth and had similar survival rates compared to PBS controls. However, mice that received the DC vaccine (DCs pulsed with TMVs incorporated with GPI-IL-12 and GPI-GM-CSF) showed a significant inhibition of tumor growth compared to all other groups. Mice from the PBS control group, DC alone or Plain TMV pulsed DC groups only survived until day 35 post tumor inoculation. Treatment with the DC vaccine resulted in a significant improvement in survival (day 35 versus day 57) compared to controls (FIG. 4).

### DC Vaccine Inhibits 4T1 Tumor Growth, Reduces Lung Metastasis, and Improved Immune Profile of Tumors

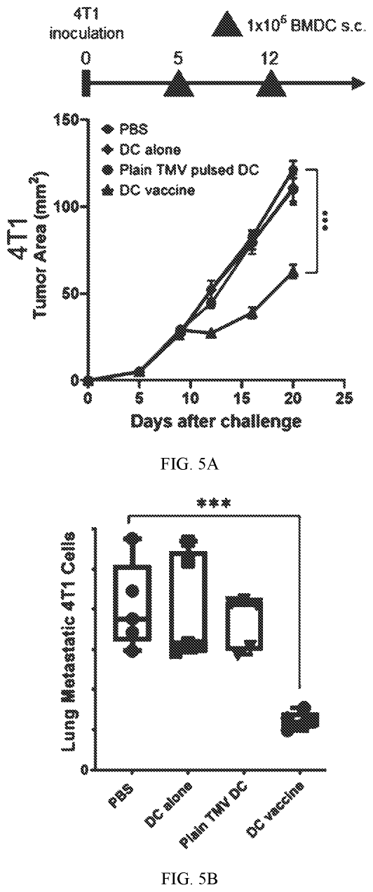

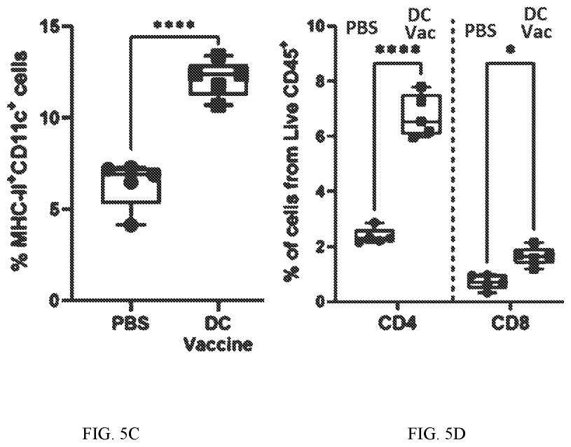

The 4T1 TNBC model represents a highly metastatic cancer that lacks a defined target antigen. Mice were challenged with 4T1 cells and DCs pulsed with 4T1 TMV vaccine were injected on the opposite flank after five days of tumor inoculation. Similar to the observations in the D2F2/E2 model, only treatment with DCs pulsed with 4T1 TMVs containing both GPI-ISMs (DC vaccine) resulted in a significant inhibition of tumor growth (FIG. 5A). Given that the 4T1 model is spontaneously metastatic, the metastatic burden in the lungs of tumor bearing mice was quantified after 20 days of tumor growth. Treatment with the DC vaccine resulted in a 2 to 3-fold reduction in metastatic 4T1 cells in the lungs, compared to PBS controls, DC alone, or plain TMV pulsed DC therapy (FIG. 5B). To characterize the immune infiltrates of 4T1 tumors after therapy with TMV vaccine, tumors were resected and processed as described in the methods section to isolate tumor infiltrating cells. Flow cytometry analysis showed a 2-fold increase of MHC-II+CD11c+DCs in 4T1 tumors after mice received the DC vaccine compared to PBS controls (FIG. 5C). Further, DC vaccine treatment resulted in a significant increase in CD4 and CD8 T cells in the tumor compared to PBS controls (FIG. 5D).

### Bone Marrow-Derived Dendritic Cell (BMDC) Production

BMDCs were generated from femurs of female BALB/c mice. Bone marrow was flushed using RPMI-1640 medium with a 22 G needle and syringe. Red blood cells (RBC) were lysed using RBC lysis buffer and resulting cells were cultured in complete RPMI-1640 medium containing 20 ng/mL recombinant murine GM-CSF (rmGM-CSF, BioLegend, San Diego, Calif., USA) at a density of 2×10⁵ cells/mL. The rmGM-CSF was replenished at days 3, 6 and 8 in culture and BMDCs were ready for use at day 10.

TMV Uptake, DC Pulsing, and Tumor Challenge Studies

For BMDC uptake, TMVs were labeled using the pH sensitive pHrodo Deep Red™ mammalian and bacterial cell labeling kit. TMV (50 μg) were diluted in 1 mL of cell labeling and wash buffer with 1.5 μg of pHrodo Deep Red™ dye. The mixture was incubated for 2 h at room temperature, then washed once with wash buffer, followed by a PBS wash. No fluorescence of TMVs was detected by flow cytometry with FACS buffer (PBS with 5% FBS, 0.05% sodium azide, 5 mM EDTA) at pH 7 but detected only after acidification to pH 4 with diluted HCl. The BMDCs were cultured with pHrodo™ labeled TMVs for 30, 90 or 180 min and followed by flow cytometry analysis.

For BMDC pulsing, 400,000 DCs/mL were cultured in 1 mL of media with or without 20 μg of TMVs. Lipopolysaccharide (LPS) was used as a positive control for activation at a concentration of 1 μg/mL. For D2F2/E2 challenge studies, BALB/c mice were inoculated with 2×10⁵ D2F2/E2 cells in 100 μL PBS subcutaneously (s.c.) in the hind flank. For 4T1 challenge studies, BALB/c mice were inoculated with 5×10⁴ 4T1 cells in 100 μL PBS s.c. in the hind flank. Tumor diameters were measured twice per week using a digital vernier caliper. BMDCs were pulsed with the respective D2F2/E2 or 4T1 TMVs for 24 h, followed by two rinses with PBS to remove unbound TMV. DCs were harvested and washed twice with PBS by centrifugation at 300×g for 5 min and injected s.c. into the opposite flank of the tumor at 1×10⁶ DCs per dose.

### 4T1 Lung Metastasis Assay

Lungs were collected from mice bearing 4T1 tumors after 20 days of tumor inoculation. These were processed under sterile conditions, minced, and digested in 1 mg/mL collagenase IV for 2 h at 37 C under rotation. The cell suspension was filtered and washed twice in selection media composed of complete DMEM containing 60 μM 6-thioguanine (6-TG). This treatment kills lung fibroblasts without affecting tumor cells. Once one of the wells reached confluency, all the wells were harvested and counted using a Cellometer™ T4 Auto counter.

1. A dendritic cell pulsed in vitro with tumor membrane vesicles comprising:
a tumor cell antigen on the surface of the tumor membrane vesicles;
a first fusion protein on the surface of the tumor membrane vesicles having a granulocyte-macrophage colony-stimulating factor (GM-CSF) expressed and a glycosyl phosphatidylinositol domain; and
a second fusion protein on the surface of the tumor membrane vesicles having interleukin 12 domain and a glycosyl phosphatidylinositol domain.

2. The dendritic cell of claim 1 wherein the tumor membrane vesicles (TMV) are obtained from a tumor tissue or cultured tumor cells.

3. The dendritic cell of claim 2, wherein the tumor membrane vesicles have a diameter of about 200 to 1000 nanometers.

4. The dendritic cell of claim 1, first fusion protein and second fusion protein incorporated into the tumor membrane vesicles by contacting the first fusion protein and second fusion protein with the tumor membrane vesicle.

5. The dendritic cells of claim 1, wherein the tumor antigen is human epidermal growth factor receptor 2 (HER2).

6. The dendritic cells of claim 1 derived from bone marrow.

7. A method of treating cancer comprising administering an effective amount of dendritic cell pulsed in vitro with the tumor membrane vesicles of claim 1 to a subject in need thereof.

8. The method of claim 7, wherein the tumor membrane vesicles are obtained from tumor tissue or cultured tumor cells from the subject to be treated.

9. The method of claim 7, wherein the tumor membrane vesicles are obtained from tumor tissue or cultured tumor cells not from the subject to be treated.

10. The method of claim 7, wherein the cancer is breast cancer.

11. The method of claim 7, wherein the subject is diagnosed with a HER2 positive cancer.

12. The method of claim 7, wherein the subject is diagnosed with triple negative breast cancer.

13. The method of claim 7, wherein the dendritic cell pulsed in vitro with tumor membrane vesicles are administered in combination with another chemotherapy agent.
